(19) United States
(12) Patent Application Publication
(10) Pub. No.: US 2012/0289836 A1
(43) Pub. Date: Nov. 15, 2012

(54) AUTOMATIC REAL-TIME DISPLAY SYSTEM FOR THE ORIENTATION AND LOCATION OF AN ULTRASOUND TOMOGRAM IN A THREE-DIMENSIONAL ORGAN MODEL

(76) Inventors: Osamu Ukimura, Pasadena, CA (US); Masahiko Nakamoto, Pasadena, CA (US); Yoshinobu Sato, Suita (JP); Norio Fukuda, Kunisaki (JP)

(21) Appl. No.: 13/467,913

(22) Filed: May 9, 2012

Related U.S. Application Data
(60) Provisional application No. 61/518,899, filed on May 12, 2011.

(51) Int. Cl. A61B 8/12

(52) U.S. Cl. 600/463

(57) ABSTRACT

An automatic real-time display system of the orientation and location of an ultrasound tomogram in a three-dimensional organ model based on the tracking of free-hand manipulation of an ultrasound probe having an AHRS sensor to acquire an entire three-dimensional volume data of an organ and real-time display to visualize the orientation and location of the ultrasound tomogram in three dimensions.

### CROSS-REFERENCE TO RELATED APPLICATION(S)

This application claims priority to U.S. Provisional Application No. 61/518,899, filed May 12, 2011, the contents of which are incorporated herein by reference.

### BACKGROUND

Ultrasound is the most popular imaging modality at a patient bed-side, and is safe for both patients and clinicians because there is no radiation exposure during its use. Definitive diagnosis of prostate cancer is made by pathological diagnosis of biopsy specimens, which are generally sampled by a transrectal ultrasound (TRUS) guided needle biopsy. Currently, a bi-plane TRUS probe which allows simultaneous display of both axial and sagittal scanning of the prostate is available to enhance the precision of the imaging, although regular urologists generally need significant experience to use this probe functionally.

An important shortcoming of current prostate biopsies, performed by most regular urologists (not by an expert), is that the biopsy procedures are image-blind procedures, in other words, they do not target or search any TRUS-visible abnormal lesions, due to the difficulty of interpreting abnormalities in TRUS imaging. Importantly, studies have found that cancers detected by image-guided targeted biopsies are of higher grade and larger volume; therefore they are more clinically important than those of image-blind biopsies. Since such image-guidance to visible lesions can facilitate needle delivery to the center of cancers or geometrically specific sites where the likelihood of cancer is higher, image-guided targeting should be considered as a key technique in maximizing the detection of cancer as well as minimizing the taking of unnecessary numbers of biopsy cores.

However, a limitation of TRUS-imaging is that it is operator dependent, requiring a significant learning curve. If a regular urologist used a single TRUS image, the orientation of the current ultrasound (US) imaging in the three-dimensional volume data of the prostate (i.e. which section of the organ in the three-dimensional prostate is now imaged by the current two-dimensional US image) is not easily recognized likely losing the three-dimensional orientation of the imaging section.

Spatial location of the TRUS probe can be tracked using either a magnetic tracking system or an optical tracking system, the former requires wired-magnetic sensors and manipulation of the US probe within the limited magnetic fields which is generated surrounding the patient; while the latter requires three or more optical markers attached to the probe, and the attached markers need to be tracked within the limited view-fields of an optical infra-red sensor camera. A third technique to track the location of the US probe is by mechanical control of the orientation and location of the US probe by a robotic arm; however, since current mechanical manipulation is a complicated and difficult procedure most suitable by a clinician's free-hand easy-handling manipulation, the robotic control of the US probe still requires further improvements.

Consequently a need exists for an improved ultrasound system for image-guided prostate biopsy procedures which addresses the limitations of previous ultrasound systems and methods.

### SUMMARY OF THE INVENTION

The present invention is directed to an automatic real-time display system of the orientation and location of an ultrasound tomogram in a three-dimensional organ model which can be displayed in real-time in a three-dimensional organ model according to the actual orientation and location of a transrectal ultrasound bi-plane probe during a clinicians free-hand manipulation of the probe. The system of the present invention includes an ultrasound machine having a transrectal ultrasound probe which may include an attitude heading reference system (AHRS) sensor attached to the ultrasound probe and a computer having software with the ability to reconstruct a three-dimensional model of the organ based on tracking the free-hand manipulation of the ultrasound probe to acquire the entire three-dimensional volume data of the organ, and a display screen to visualize the orientation and location of the tomogram in a three-dimensional display. The software can also reconstruct the three-dimensional organ model without AHRS data.

The AHRS sensor provides enhanced accuracy in the functions of a vertical gyro and a directional gyro to provide measurement of roll, pitch, heading (azimuth) angles, and attitude information of the probe. Advantages of using AHRS for tracking the US probe include (i) the AHRS system is a less expensive system than other previously used tracking systems such as magnetic, optical, or robotic tracking systems, (ii) accuracy of the AHRS system will not be disturbed either by the metals in the surgical field, such as by a metallic surgical bed; as the disturbance of magnetic field by metals is the major disadvantage in the magnetic tracking system or by the obstruction against the view-field of the optical camera due to the intra-operative dynamic movements of either clinician's hands or angle of the US probe, and (iii) AHRS is a small, single sensor able to track the orientation and location of US probe in an unlimited condition except for as long as the wire of AHRS reaches to the hardware; therefore, the use of AHRS will allow easier, quicker, and more smooth free-hand manipulation of the US probe for clinicians compared to the existing other tracking technologies mentioned above.

As such, during free-hand manipulation of the US probe, the invention of the automatic real-time display system of the orientation and location of the US tomogram in the three-dimensional organ model improves the quality of the prostate biopsy procedure.

### BRIEF DESCRIPTION OF THE DRAWINGS

FIG. 1 is a schematic diagram of the automatic real-time display system of the orientation and location of an ultrasound tomogram in a three-dimensional organ model of the present invention;

FIG. 2 is a flow-chart of the software of the system of FIG. 1;

FIG. 3 is a schematic illustration of the three-dimensional ultrasound image of the present invention;

FIG. 4 is a diagram of the Y-Z cross-section of a three-dimensional ultrasound image of

FIG. 3;

FIG. 5 is a schematic diagram of the coordinate systems of the ultrasound images; and

FIG. 6 is a schematic illustration of the visualization of a three-dimensional organ model in the ultrasound image planes.

### DETAILED DESCRIPTION OF THE DRAWINGS

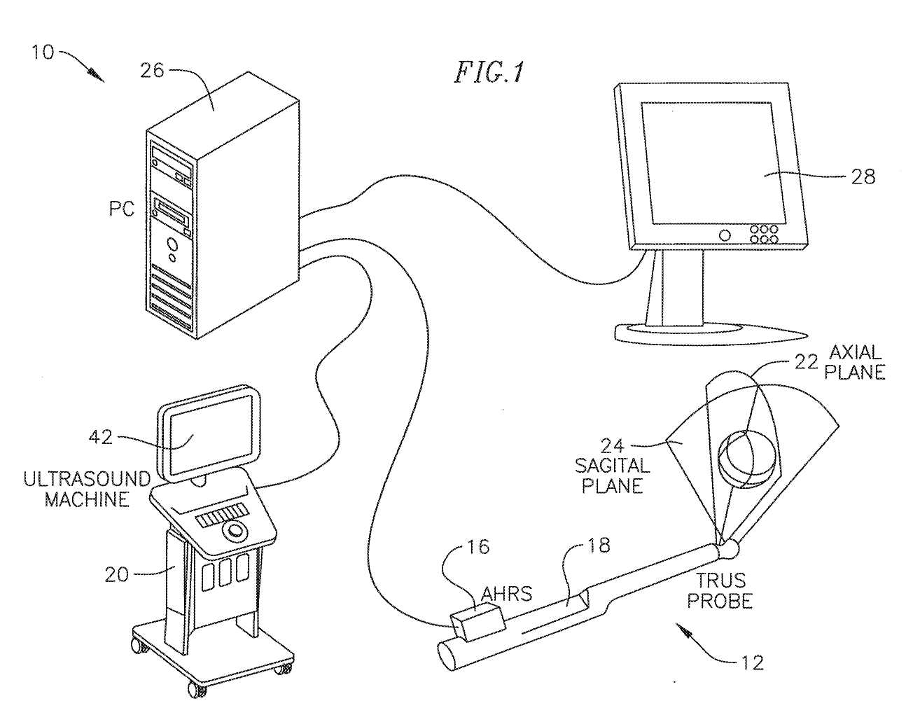

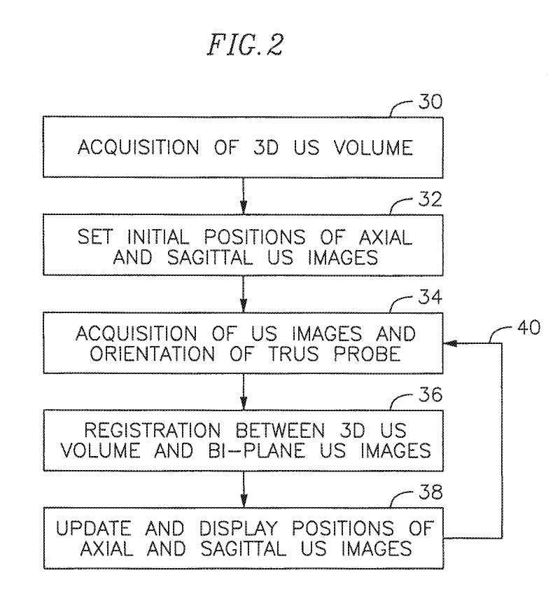

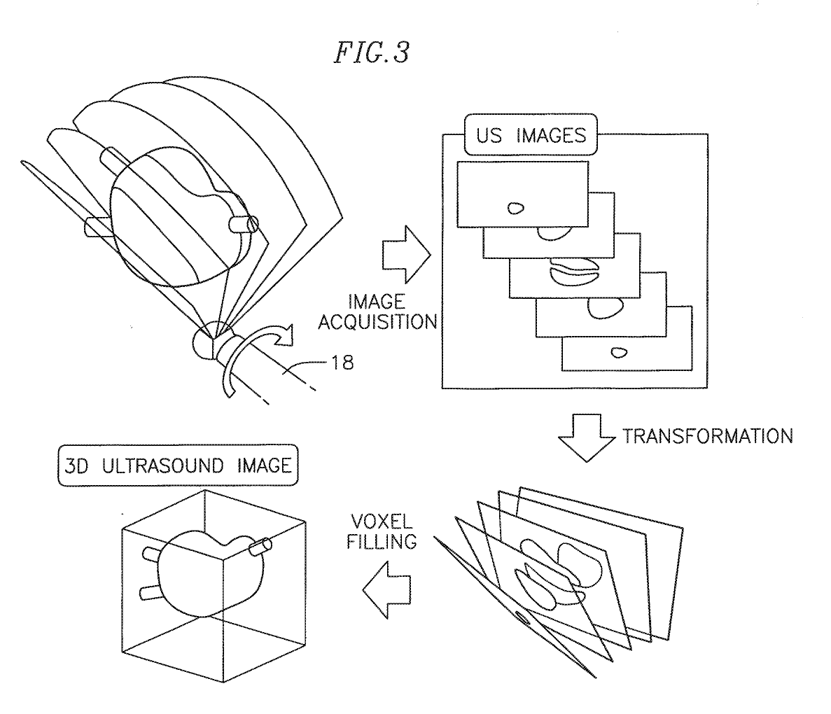

FIG. 1 illustrates an automatic real-time display system of the orientation and location of the US tomogram in a three-dimensional organ model 10 of the present invention. The automatic real-time display system 10 includes unique hardware 12 incorporating an attitude heading reference system (AHRS), and computer-software 14 (FIG. 2) to support the system having the ability to reconstruct a three-dimensional model of the organ (prostate) based on tracking of the freehand manipulation of an ultrasound probe to acquire the entire three-dimensional volume data of the organ (prostate), and an unique real-time display to visualize the orientation and location of TRUS tomogram in three dimensions.

The invention utilizes a unique tracking system which involves the use of an AI-IRS sensor 16 which provides the functions of a vertical gyro and a directional gyro to provide measurement of roll, pitch, heading (azimuth) angles, and attitude information. A wired or wireless AHRS sensor 16 is attached and fixed to a TRUS probe 18, externally. The AHRS sensor fixed to the TRUS probe measures its orientation and acceleration. The AHRS sensor 16 can be fixed on the TRUS probe 18 by being either attached on the surface of the TRUS probe, or built into the inside of the TRUS probe. The probe 18 is a bi-plane transrectal ultrasound (TRUS) probe which is electrically connected to an ultrasound machine 20. Two ultrasound images on the orthogonal planes, namely the axial 22 and the sagittal 24 planes can be acquired by the probe and displayed simultaneously on the ultrasound machine. The AHRS sensor provides information of orientation of three axes and acceleration of three axes to a computer (PC) 26 which includes a graphics processing unit (GPU). The ultrasound machine is also electrically connected to the computer.

The ultrasound images acquired by the ultrasound machine 20 are transferred to the PC 26 in real-time. The AHRS sensor 16 fixed to the TRUS probe 18, which measures its orientation and acceleration, also transfers the measured data to the PC in real-time. At the PC, the positions of the axial and sagittal planes of the ultrasound images are estimated by using the captured ultrasound images and measured data by the AHRS sensor, and then they are displayed on a monitor 28.

The computer 26 includes software 14 to reconstruct a three-dimensional model of the organ based upon the tracking of the free-hand manipulation of the ultrasound probe. The software as schematically illustrated in FIG. 2 includes five steps:

1. Acquisition of three-dimensional ultrasound image.

2. Determination of initial positions of axial and sagittal planes.

3. Acquisition of bi-plane ultrasound images and measurement of orientation and acceleration of the ultrasound probe.

4. Estimation of position of axial and sagittal planes by registration between the three-dimensional ultrasound image and the bi-plane ultrasound images.

5. Update display.

At the first step 30, a three-dimensional ultrasound image (3D US) is reconstructed from a series of two-dimensional sagittal ultrasound images and orientation data measured by the AHRS sensor, which are acquired while rotating the TRUS probe, or through a series of only two-dimensional axial and sagittal ultrasound images without orientation data measured by the AHRS, which are acquired while moving the TRUS probe in forward and backward directions. The reconstructed 3D US is employed as the reference volume as the fourth step. At the second step 32, the initial positions of the axial and sagittal planes for registration between them and the 3D US are determined. The first and second steps are preparation for the real-time position estimation (steps three to five). At the third step 34, ultrasound images on the axial and sagittal planes are acquired and orientation and acceleration of the TRUS probe are measured. At the fourth step 36, by using these data, registration between the 3D US and acquired ultrasound images are performed, and then the current position of the US images on the prostate are determined. At the fifth step 38, the US plane models are located at the obtained position on the three-dimensional prostate model.

The third to fifth steps are a real-time visualization process of the current positions of the US image planes which a physician is watching, and these steps are repeated 40 during the intervention.

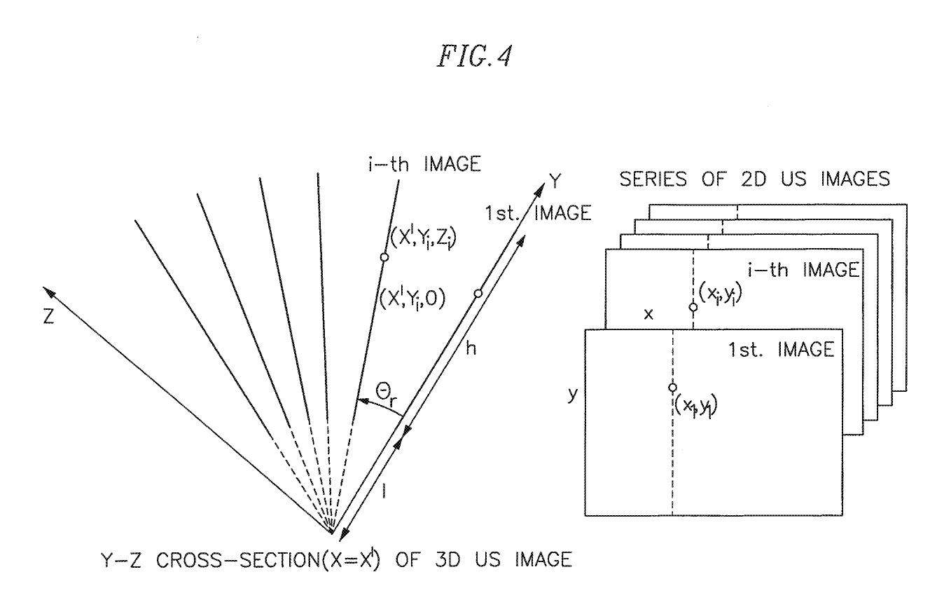

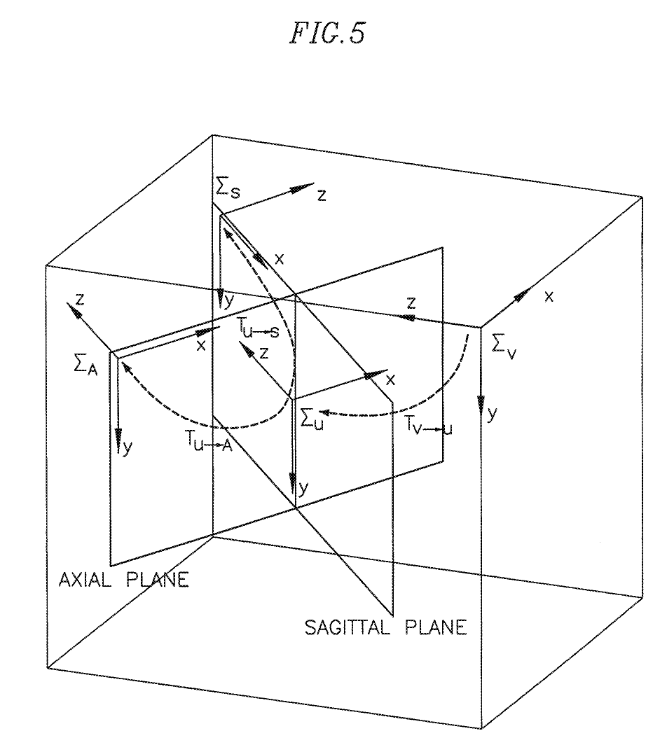

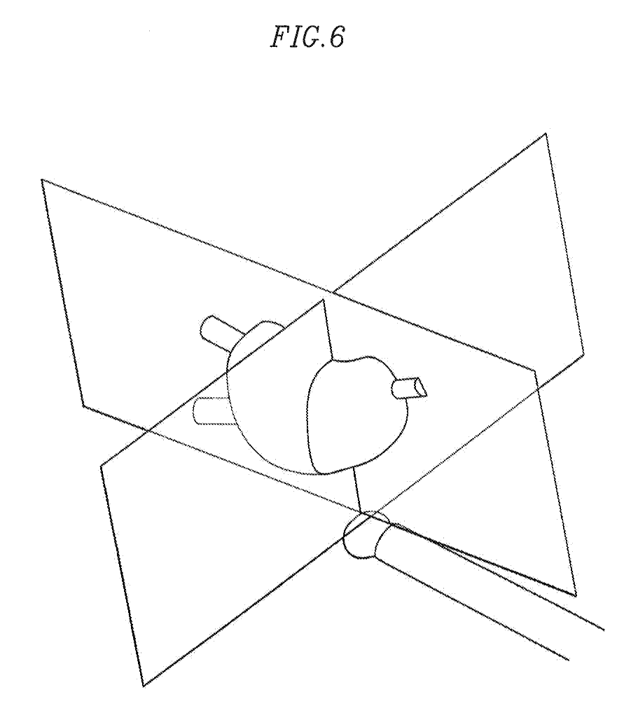

In the first step 30, acquisition of three-dimensional ultrasound image, a 3D US is reconstructed from a series of US images acquired by rotating the TRUS probe 18 and orientation of the TRUS probe measured by the AHRS sensor as shown in FIG. 3. As shown in FIG. 4, the number of acquired US images is represented by i-th (for example, when i-th is 1st, 2nd, 3rd or 4th, i-th US image means the 1st, 2nd, 3rd , or 4th US image, respectively). The pixel on i-th US image whose coordinate is (x, y) is mapped to the position (X, Y, Z) on the three-dimensional US image coordinate system by the following transformation:

$$( \begin{matrix}X \\ Y \\ Z \\ 1\end{matrix} ) = ( \begin{matrix}1 & 0 & 0 & 0 \\ 0 & cos {θ}_{i} & - sin {θ}_{i} & 0 \\ 0 & sin {θ}_{i} & cos {θ}_{i} & 0 \\ 0 & 0 & 0 & 1\end{matrix} ) ( \begin{matrix}s & 0 & 0 & 0 \\ 0 & - s & 0 & l + sh \\ 0 & 0 & 1 & 0 \\ 0 & 0 & 0 & 1\end{matrix} ) ( \begin{matrix}x \\ y \\ 0 \\ 1\end{matrix} )$$

where θᵢ, l, s and h are a rotation angle of the TRUS probe, distance between the US image and the TRUS probe, pixel size of the US image and height of the US image, respectively. l, s and h are determined by calibration which is performed beforehand (. 4). A corresponding voxel for each pixel is determined by this transformation, and then the pixel value is filled in the corresponding voxel. If multiple pixels corresponds to one voxel, an average pixel value among those pixels is filled in the voxel. After this process is performed for all acquired US images, hole filling is performed to eliminate empty voxels.

In the second step 32, determination of initial positions of ultrasound images, in order to estimate positions in step 4 accurately, initial positions of the real-time US images to the estimation algorithm have to be provided. Initial positions are determined by finding correspondence between the three-dimensional US image and the real-time US images.

In the third step 34, acquisition of real-time two-dimensional ultrasound image and measurement of orientation and acceleration of TRUS probe, the real-time two-dimensional US images on the axial and sagittal planes are displayed on the monitor 42 of the US machine 20. The video output of the US machine is connected to a frame grabber board in the PC 26, and then the US images are digitized and captured in real-time. In synchronization with image capture, orientation and acceleration of the TRUS probe are measured by AHRS sensor.

In the fourth step 36, position estimation of real-time two-dimensional ultrasound image, the positions of real-time two-dimensional US images are estimated by registration between the three-dimensional US image and the real-time two-dimensional US images. As shown in . 5, let Σ_{V} , Σ_{U}, Σ_{A} and Σ_{S} be coordinate systems of the three-dimensional US image, two-dimensional US images, axial plane and sagittal plane, respectively. Σ_{V}, Σ_{A} and Σ_{S} represent the origin and direction of each image. Σ_{U} is the coordinate system to handle the axial and sagittal planes as one object. Position of Σ_{U} is the center of gravity of the axial plane, and the directions of its axes are parallel to those of Σ_{A}. Registration is that to determine rigid transformations from Σ_{V} to Σ_{A} and Σ_{S}, and these transformations are defined as 4×4 matrices, T_{V→A} and T_{V→S}. Since T_{U→A} and T_{U→S} are fixed transformation and do not change during estimation, they are determined by prior calibration, and T_{V→A} and T_{V→S} can be described by using them as ^{T}_{V→A}=T_{V→U}T_{U→A} and T_{V→S} =T_{V→U}T_{U→S}, respectively. Therefore, estimation of T_{V→U} is performed instead of estimation of T_{V→A} and T_{V→S}.

Registration is performed by minimizing difference between captured two-dimensional US images and corresponding slices clipped from the three-dimensional US image. This process is formulated as follows:

$${\tilde{T}}_{U \to V} = arg {min}_{{T}_{U \to V}} \{ S ( {I}_{a} , {F}_{a} ( {I}_{V} , {T}_{V \to U} {T}_{U \to A} ) ) + S ( {I}_{S} , {F}_{S} ( {I}_{V} , {T}_{V \to U} {T}_{U \to S} ) \} ,$$

where S(I, J) is a function to measure the difference between image I and image J. The sum of squared difference, normalized cross correlation and mutual information are employed as a measure of image difference. F(I,T) is a function to clip a two-dimensional image slice located at T from a three-dimensional image I. If the AHRS sensor is equipped on the TRUS probe, an orientation data measured by the AHRS sensor can be used for the estimation. T_{U→V} can be divided to rotational part R and translational part t as

$${T}_{U \to V} = ( \begin{matrix}R & t \\ 0 & 1\end{matrix} ) .$$

Since the rotational part is measured by the AHRS sensor, only the translational part is estimated by registration. The Powell method or the Levenberg-Marquardt method is employed for minimization. The position obtained at Step 2 is used as the initial position at the first estimation, and the previous result is used at after that.

In the fifth step 38, update of displayed information, the prostate region is segmented from the three-dimensional US image and then a three-dimensional prostate model is reconstructed beforehand. On the three-dimensional prostate model, the axial and sagittal planes are located at the estimated position as shown in .6. The color and opacity of these models can be changed by the operator. The captured US images can be mapped onto these planes. Furthermore, in order to confirm correctness of registration, the real-time US images and the corresponding slice clipped from the three-dimensional US image can be displayed.

Although the present invention has been described and illustrated with respect to an embodiment thereof, it should be understood that the invention is not to be so limited as changes and modifications can be made herein which are within the scope of the claims as hereinafter recited.

What is claimed is:

1. An automatic real-time display system for the orientation and location of an ultrasound tomogram in a three-dimensional organ model comprising:
an ultrasound machine;
an ultrasound probe; and
a computer having software configured to reconstruct a three-dimensional model of an organ based upon tracking a free-hand manipulation of the ultrasound probe to acquire the entire three-dimensional volume data of the organ and having a real-time display to visualize an orientation and location of an ultrasound tomogram in three dimensions.

2. The system of claim 1 wherein the ultrasound probe is a bi-plane transrectal ultrasound probe.

3. The system of claim 1 wherein the ultrasound probe includes an AHRS sensor which is connected externally on the probe and is wired to the computer.

4. The system of claim I wherein the ultrasound probe includes an AHRS sensor which is a wireless sensor within the ultrasound probe.

5. A method for real-time display of orientation and location of an ultrasound tomogram in a three-dimensional organ model comprising the steps of
acquisition of a three-dimensional ultrasound image;
determination of initial positions of axial and sagittal planes;
acquisition of bi-plane ultrasound images and measurements of orientation and acceleration of an ultrasound probe;
estimation of position of axial and sagittal planes by registration between the three-dimensional ultrasound image and the bi-plane ultrasound images; and
updating a displayed three-dimensional image.

6. The method of claim 5 wherein the step of acquisition of a three-dimensional ultrasound image is through reconstruction from a series of two dimensional sagittal ultrasound images and orientation data measured by an AHRS sensor connected to an ultrasound probe which are acquired by rotating the ultrasound probe.

7. The method of claim 5 wherein the step of acquisition of a three-dimensional ultrasound image is through reconstruction from a series of two-dimensional axial and sagittal ultrasound images which are acquired by movement of an ultrasound probe in a forward and backward direction.

8. The method of claim 5 wherein the steps of acquisition of bi-plane ultrasound images and measurement of orientation and acceleration of an ultrasound probe, estimation of position of axial and sagittal planes and updating a displayed three-dimensional image are in real-time position estimation.

9. A medical device comprising:
an ultrasound machine;
an ultrasound probe connected to the ultrasound machine;
an AHRS sensor connected to the ultrasound probe;
a computer connected to the ultrasound machine and the AHRS sensor configured to display a three-dimensional ultrasound tomogram.

10. The device of claim 9 wherein the ultrasound probe is a bi-plane transrectal ultrasound probe.

11. The device of claim 9 wherein the AHRS sensor is connected externally on the probe and is wired to the computer.

12. The device of claim 9 wherein the AHRS sensor is a wireless sensor within the ultrasound probe.

13. The device of claim 9 wherein the computer is configured to display a three-dimensional ultrasound tomogram by software.
